(19) United States
(12) Patent Application Publication
(10) Pub. No.: US 2020/0338051 A1
(43) Pub. Date: Oct. 29, 2020

(54) NON-CLINICAL LIQUID FORMULATIONS OF PAN-JAK INHIBITOR

(71) Applicant: Janssen Pharmaceutica NV, Beerse (BE)

(72) Inventor: Michele C. Rizzolio, San Diego, CA (US)

(21) Appl. No.: 16/857,663

(22) Filed: Apr. 24, 2020

Related U.S. Application Data
(60) Provisional application No. 62/837,849, filed on Apr. 24, 2019.

(51) Int. Cl. A61K 31/437; A61K 47/40

(52) U.S. Cl. CPC A61K 31/437; A61K 47/40

(57) ABSTRACT

Pan-JAK inhibitor preclinical formulations.

The considerable resources and efforts that are dedicated to the discovery of new chemical entities for drug development may be adversely impacted by limitations in the formulation work that is needed to have the chemical entities of interest in forms and media that can be used for the non-clinical and clinical work that is needed in drug development. The inability to come up with an adequate development formulation, or the inability to be able to do so with adequate expediency, might lead to the abandonment of a new chemical entity from further development due to absorption, distribution, metabolism, and elimination issues when such entity might have otherwise been regarded as a promising candidate to address a medical need. Animal experiments and the finding of appropriate formulations to be administered in the same can play an important role in drug development. Furthermore, pre-clinical formulation work can provide important information to identify the limitations and development potential of chemical entities in later stages, particularly those that would involve administration into human beings.

In the multi-step approach to drug discovery and development, formulations are needed for non-clinical testing, such as for non-clinical toxicology studies. Embodiments of formulations according to this invention concern formulations for use during the development stage between the new chemical entity synthesis and first-in-human clinical studies. Because formulations for non-clinical use are also needed in later development stages, embodiments of formulations according to this invention also concern formulations that, while still not envisaged for use with human beings, are prepared for their use in post first-in-human phases of development. The terms “pre-clinical formulation” and “non-clinical formulations” as used herein encompass, unless indicated otherwise, formulations envisaged for use before the first-in-human development stage and also formulations envisaged for use at other development stages that do not entail administration of such formulations to human beings.

Multiple factors condition pre-clinical formulation work, and reference materials are available setting forth suggested protocols and formulation strategies. However, some compounds have features such that available reference material does not provide teachings or suggestions that can be implemented in a specific development work with reasonable expectations of success, hence the need for inventive formulation work. See, for example, S. M. Shah, el al., Preclinical Formulations: Insight, Strategies, and Practical Considerations, AAPS PharmSciTech 15(5), 1307-23 (2014), S. Neervannan, Preclinical formulations for discovery and toxicology: physicochemical challenges, Expert Opinion on Drug Metabolism & Toxicology 2(5), 715-731 (2006); S. Gopinathan, et. al., Development and application of a high-throughput formulation screening strategy for oral administration in drug discovery, Future Med Chem 2(9), 1391-98 (2010); and T. Loftson, et al, Preparation of solid drug/cyclodextrin complexes of acidic and basic drugs, Pharmazie 59, 25-29 (2004), and references therein for reviews on pre-formulation work and the challenges that it still presents.

The plurality of factors that play an important role in pre-clinical formulation work include those that are summarized as follows:

Vehicle and route of administration, solubility and related features of absorption and bioavailability;

consideration of the administration route that is envisaged for human beings;

knowledge of toxicology, pharmacokinetics, analytical techniques, and chemical and physical characterization;

maximizing exposure or efficacy by exposing the site of action to a high number of active chemical entities to identify adverse effects that the chemical entity might cause, an important goal in early animal studies to further the understanding of the pharmacokinetics, pharmacodynamics and toxicological signals together with target biological response;

formulation excipient, delivery system, and thorough understanding of physicochemical parameters;

solubility and solution stability through solubilization techniques and aqueous monophasic solution preparation and stabilization techniques, sometimes with the aid of external sources of energy, such as sonic waves, heat or vortexing with the active chemical entity either solubilized or suitably dispersed: choice of solvent and sometimes co-solvents to address low aqueous solubility can be additional factors;

when pursuing drug/cyclodextrin complexes, achieving a sufficient drug concentration with acceptable amounts of cyclodextrin that enable solubilization while avoiding instabilities such as precipitations or chemical changes of part of the drug;

need and amount of formulation ingredients, including vehicles, and if needed, safety of the same while achieving appropriate uniformity of content and ease of manufacture and administration,

salt disproportionation or change to low solubility polymorphs, that could lead to undesirable precipitation;

chemical entity batch and purity monitoring and form selection and characterization that do not lead to inconsistencies, a selection that for some chemical entities can be a complex process in light of the plurality of forms in which the chemical entity presents itself, sometimes being in a large number of forms;

limitations in the amount of available chemical entity and time requirements for testing of the same; and

effective decision making on the basis of pre-clinical work that is as streamlined and well-understood as possible.

2-(1-((1r,4r)-4-(cyanomethyl)cyclohexyl)-1,6-dihydroimidazo[4,5-d]pyrrolo[2,3-b]pyridin-2-yl)-N-(2-hydroxy-2-methylpropyl)acetamide (“drug substance”) is a pan-JAK inhibitor that can be prepared in a large plurality of solid forms. It has been shown to have low systemic exposure. After both oral and intra-colonic dosing, the drug substance concentrations in the colon were found to be much higher than those in the plasma as described in US 2018/0170931 A1, published Jun. 21, 2018, (“the '931 publication”), Table 1a, p. 11 and Table 1b, p. 12 and ¶ [0132], p. 12. In in vitro studies, the drug substance demonstrated potent enzymatic activity when tested at JAK1, JAK2, JAK3 and TYK2 as shown in the '931 publication, Table 4, p. 27. The drug substance also showed cellular activity in the inhibition of STAT phosphorylation when tested in peripheral blood mononuclear cell (PBMC) using stimuli IL-2, INF-α and GM-CSF which measured the inhibition of phosphorylation of STAT5, STAT4 and STAT5, respectively, as shown in the '931 publication, Table 5, p. 29. Preparation and solid form isolation of the drug substance is performed, for example, as described in the '931 publication, example 1, p. 16, intermediate 1, p. 14, intermediate 2, p. 15, and intermediate 3, p. 15, polymorph screening example, pp. 22-26 and figures and tables referred to therein. References in this application to the '931 publication are incorporated herein by reference in their entirety.

There is a need for pre-clinical formulation work for the drug substance that is under development consideration for certain indications. Some conventional approaches to pre-clinical formulations of the drug substance led to unacceptable formulations. In contrast, the drug substance could be formulated in other ways that unexpectedly led to formulations of the same that were suitable as pre-clinical formulations. Some of the following examples illustrate formulation embodiments that were prepared according to conventional methodologies, but that did not yield acceptable pre-clinical formulations. Other examples illustrate embodiments of this invention that led to acceptable pre-clinical formulations of the drug substance as aqueous solutions for uses that included GLP toxicology studies.

### Instrumentation and Methodology

### List of Abbreviations

mAU milli-absorbance units
HP-β-CD hydroxypropyl-β-cyclodextrin
HPLC high performance liquid chromatography
min minute
mg milligram
mL milliliter
pKa acid dissociation constant
PXRD powder X-ray diffraction
SBE-β-CD sulfonylbutylether-β-cyclodextrin

An Agilent model 1100 equipped with a Diode Array Detector was used to assess the purity and/or determine the content of formulation and stability samples of the drug substance. The HPLC method is displayed in Table 1.

TABLE 1
Features of the HPLC method for analyzing the drug substance

| Column | Xbridge C18 3.5μ, 150 × 4.6 mm | |
|---|---|---|
| Mobile phase A | 10 mM ammonium acetate in 9:1 water/methanol | |
| Mobile phase B | 10 mM ammonium acetate in 100% methanol | |
| Gradient phase | time (min) | % mobile phase B |
| | 0.00 | 10.0% |
| | 19.00 | 100.0% |
| | 20.00 | 100.0% |
| | 20.10 | 10.0% |

| | |
|---|---|
| Flow rate | 1.00 mL/min |
| Detection | 254 nm |
| wavelength | |
| Column | 40.0° C. |
| temperature | |

Powder X-ray Diffraction technology was used to obtain PXRD patterns of the drug substance, as shown in FIG. 6, tracing labeled as ‘1s’, in the '931 publication.

All the formulations were prepared using drug substance in form 1s as a free base.

Various concentrations were evaluated, ranging from 1 mg/mL to 200 mg/mL by weighing solid drug substance into a clear glass vial and diluting with vehicle to form a drug substance-vehicle mixture. The vehicle used for formulations consisted of 20% HP-β-CD or 30% SBE-β-CD in water. Because of slight changes in the vehicle volume upon addition of the drug substance and acidification, the cyclodextrin concentration in the examples of formulations given herein was slightly less than 20% (for HP-β-CD-containing solutions, and such examples of formulations are characterized as having an HP-β-CD concentration that does not exceed 20%) or 30% (for SBE-β-CD-containing solutions, and such examples of formulations are characterized as having a SBE-β-CD concentration that does not exceed 30%). Resulting mixtures were acidified to a pH between 2 to 3 using HCl, sulfuric, or phosphoric acid, (which solution are referred to herein as acidified HP-β-CD or acidified SBE-β-CD, as the case may be) and then sonicated until all was in solution. Once in solution, vials were covered in foil to protect from light exposure. Table 2 lists solution characteristics of various embodiments.

To provide a more concise description, some of the quantitative expressions given herein are not qualified with the term “about”. It is understood that whether the term “about” is used explicitly or not, and unless specified otherwise, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

Concentrations that are given as percentages refer to mass ratios, unless indicated differently.

TABLE 2

| | | | | Drug | |
|---|---|---|---|---|---|
| | | | | substance | |
| | | | | concentration | |
| Embodiment | Vehicle | Acid | pH | (mg/mL) | |
| Example 6 | 20% HP-β-CD in H₂O | HCl | 3.93 | 1 | mg/mL |
| | | (6N) | | | |
| Example 1 | 20% HP-β-CD in H₂O | HCl | 2.97 | 200 | mg/mL |
| | | (6N) | | | |
| Example 2 | 20% HP-β-CD in H₂O | HCl | 1.77 | 200 | mg/mL |
| | | (6N) | | | |
| Example 3 | 30% SBE-β-CD in H₂O | HCl | 3.07 | 200 | mg/mL |
| | | (6N) | | | |
| Example 4 | 20% HP-β-CD in H₂O | H₂SO₄ | 1.91 | 200 | mg/mL |
| | | (98%) | | | |
| Example 5 | 20% HP-β-CD in H₂O | H₃PO₄ | 2.14 | 200 | mg/mL |
| | | (85%) | | | |

Embodiments of formulations were evaluated for physical and chemical stability. Each formulation was evaluated under refrigerated conditions and protection from light for varying lengths of time. After establishing refrigerated stability, each formulation was exposed to thermal cycling which involved rapid changes in temperature (from ambient temperature to 4° C.) with vigorous stirring under both conditions. Samples were stirred at 4° C., and precipitation was observed in the H₂SO₄-containing and HCl-containing samples. Samples that resulted in precipitation were centrifuged in order to separate the solids from supernatant liquid. Solids were characterized via PXRD and the supernatant was analyzed via HPLC.

Complexation and pH control, alone or in combination, are used as solubilization techniques in drug formulation studies. See, for example, P. Li, et al., Combined Effect of Complexation and pH on Solubilization, J. Pharmaceutical Sciences, 87(12), 1535-37 (1998) (deriving a formula for the total concentration of drug in both ionized and un-ionized forms in terms of the free un-ionized drug concentration, the drug pKa, the solution pH, the un-ionized drug complexation constant, the ionized drug complexation constant, and the total concentration of complexation ligand). The expression to calculate the total concentration of drug as derived by Li, et al., however, requires the knowledge of thermodynamic constants that are not always known, such as the ionized and un-ionized drug complexation constants. Furthermore, such expression is for the total drug concentration on the basis of thermodynamic quantities, with no information on specific drug reactivity, and stability. This equation does not teach or suggest how to find what pH controlling agent and what complexation ligand will lead to an acceptable pre-clinical formulation for any given drug substance at a sufficiently high drug substance concentration (for example, in the range of 100 mg/mL to 250 mg/mL, or at about 200 mg/mL) that remains stable for sufficiently long time at usual storage conditions. This is something that is subject to a plurality of unpredictable factors rather than to just result-effective variables that would be susceptible of optimization. Furthermore, successful use of certain pH controlling agents and complexation ligands with some drugs, do not provide a reason for an expectation of successful results when used for pre-formulation work with different chemical entities. This is more so when drug development concerns new chemical entities whose properties are not fully known. It was unexpectedly found that while the formulation embodiments prepared with any of the acids HCl, H₂SO₄ and H₃PO₄ and cyclodextrin forms were stable while refrigerated (Table 3 below), only the H₃PO₄ formulations were stable in desired conditions (Table 4 below) while the drug substance was dissolved at a sufficiently high concentration. As shown in the tabular presentation of illustrative embodiments below, the only viable formulation that maintained physical and chemical stability was in 20% HP-β-CD acidified with H₃PO₄, embodiment presented as example 5.

TABLE 3
Summary of stability under refrigerated conditions

| | | | | | % | Days of |
|---|---|---|---|---|---|---|
| | | | | Precipitation? | Remaining | Stability |
| Example | Formulation | Concentration | Acid (pH) | (yes/no) | in solution | at 4° C. |
| 1 | Acidified HP-β-CD | 100 mg/mL | HCl (2) | No | 100 | 12 |
| 2 | Acidified HP-β-CD | 200 mg/mL | HCl (3) | No | 100 | 7 |
| 3 | Acidified SBE-β-CD | 200 mg/mL | HCl (3) | No | 100 | 45 |
| 4 | Acidified HP-β-CD | 200 mg/mL | H₂SO₄ (2) | No | 100 | 21 |
| 5 | Acidified HP-β-CD | 200 mg/mL | H₃PO₄ (2) | No | 100 | 21 |

Upon temperature cycling, formulations containing HCl and H₂SO₄ resulted in precipitation of white crystalline solids (crystallinity verified by PXRD). PXRD scan of the white precipitates were different from that of the drug substance. HPLC analysis showed that the retention time of the precipitates still matched that of the drug substance, thus verifying that the precipitate contains the same molecule as the parent. It is possible that the crystallized product is either a different polymorph of the freebase or a salt-form of the drug substance. In addition to the white precipitate, solutions acidified with HCl resulted in insoluble red particles, which were verified to be a drug substance degradation product when analyzed by mass spectrometry and HPLC analysis.

TABLE 4
Summary of formulation stability

| | | | | | Final |
|---|---|---|---|---|---|
| | | Target | Acid | Precipitation? | Concentration** |
| Example | Formulation | Concentration | (pH) | (yes/no) | (mg/mL) |
| 1 | Acidified HP-β-CD | 100 mg/mL | HCl (2) | Yes* | 82.62 |
| 3 | Acidified SBE-β-CD | 200 mg/mL | HCl (3) | Yes* | 155.07 |
| 2 | Acidified HP-β-CD | 200 mg/mL | HCl (3) | Yes* | 146.61 |
| 4 | Acidified HP-β-CD | 200 mg/mL | H₂SO₄ (2) | Yes | 70.91 |
| 5 | Acidified HP-β-CD | 200 mg/mL | H₃PO₄ (2) | No**** | 194.38*** |

*Red-colored precipitate was also observed, indicating chemical degradation
**According to HPLC analysis
***Concentrations slightly different from 200 mg/mL, such as 194.38 mg/mL, are referred to herein as concentrations of about 200 mg/mL, or as target concentrations of 200 mg/mL
****also referred to as embodiments illustrated by example 5 being stable, thus entailing concentration preservation and no precipitate formation.

Embodiments of this invention are in the form of aqueous solutions of drug substance at concentrations of up to about 250 mg/mL in 15% to 30 HP-β-CD at low pH (not exceeding 3) achieved by acidification with H₃PO₄ (these pH conditions concisely being referred to by saying that the formulation pH was achieved with phosphoric acid as acidifying agent). In some of such embodiments, the drug substance concentration is between 100 mg/mL and 250 mg/mL. Embodiments of this invention have long-term (for up to about three weeks) stability under refrigerated conditions (about 4° C.). In some embodiments, the solution pH is about 2.

### FURTHER EMBODIMENTS OF THE INVENTION

Further numbered embodiments of the invention are disclosed below.

1. An aqueous preclinical formulation comprising drug substance at a concentration of about 100 mg/mL to about 250 mg/mL, hydroxypropyl-β-cyclodextrin at a concentration that does not exceed about 30%, wherein said drug substance is 2-(1-((1r,4r)-4-(cyanomethyl)cyclohexyl)-1,6-dihydroimidazo[4,5-d]pyrrolo[2,3-b]pyridin-2-yl)-N-(2-hydroxy-2-methylpropyl)acetamide, and the formulation pH does not exceed about 3.
2. An aqueous preclinical formulation according to embodiment 1, wherein said hydroxypropyl-β-cyclodextrin concentration is about 15% to about 30%.
3. An aqueous preclinical formulation according to embodiment 2, wherein said hydroxypropyl-β-cyclodextrin concentration is about 15% to about 20%.
4. An aqueous preclinical formulation according to embodiment 1, wherein said hydroxypropyl-β-cyclodextrin concentration does not exceed about 20%.
5. An aqueous preclinical formulation according to embodiment 1, wherein said hydroxypropyl-β-cyclodextrin concentration is about 20%.
6. An aqueous preclinical formulation according to any preceding embodiment, wherein said drug substance concentration is about 100 mg/mL to about 200 mg/mL.
7. An aqueous preclinical formulation according to any preceding embodiment, wherein said drug substance concentration is about 200 mg/mL.
8. An aqueous preclinical formulation according to any preceding embodiment, wherein the aqueous preclinical formulation remains precipitate-free for at least one week at a temperature of about 4° C.
9. An aqueous preclinical formulation according to any preceding embodiment, wherein the aqueous preclinical formulation remains precipitate-free for at least two weeks at a temperature of about 4° C.
10. An aqueous preclinical formulation according to any preceding embodiment, wherein the aqueous preclinical formulation remains precipitate-free for at least three weeks at a temperature of about 4° C.
11. An aqueous preclinical formulation according to any preceding embodiment, wherein said formulation pH is about 2.
12. An aqueous preclinical formulation according to any preceding embodiment, wherein said drug substance concentration is a target drug substance concentration.
13. An aqueous preclinical formulation according to embodiment 1, wherein the aqueous preclinical formulation remains precipitate-free for at least one week at a temperature of about 4° C.
14. An aqueous preclinical formulation according to embodiment 1, wherein the aqueous preclinical formulation remains precipitate-free for at least two weeks at a temperature of about 4° C.
15. An aqueous preclinical formulation according to embodiment 1, wherein the aqueous preclinical formulation remains precipitate-free for at least three weeks at a temperature of about 4° C.
16. An aqueous preclinical formulation according to embodiment 1, wherein said formulation pH is about 2.
17. An aqueous preclinical formulation according to embodiment 1, wherein said drug substance concentration is about 200 mg/mL and said hydroxypropyl-β-cyclodextrin concentration does not exceed about 20%.
18. An aqueous preclinical formulation according to embodiment 17, wherein said formulation pH is about 2.
19. An aqueous preclinical formulation according to embodiment 17, wherein the aqueous preclinical formulation remains precipitate-free for at least one week at a temperature of about 4° C.
20. An aqueous preclinical formulation according to embodiment 17, wherein the aqueous preclinical formulation remains precipitate-free for at least two weeks at a temperature of about 4° C.
21. An aqueous preclinical formulation according to embodiment 17, wherein the aqueous preclinical formulation remains precipitate-free for at least three weeks at a temperature of about 4° C.
22. An aqueous preclinical formulation comprising drug substance at a concentration of about 200 mg/mL, hydroxypropyl-β-cyclodextrin at a concentration that does not exceed about 20%, wherein said drug substance is 2-(1-((1r,4r)-4-(cyanomethyl)cyclohexyl)-1,6-dihydroimidazo[4,5-d]pyrrolo[2,3-b]pyridin-2-yl)-N-(2-hydroxy-2-methylpropyl)acetamide, the formulation pH is about 2, and the aqueous preclinical formulation remains precipitate-free for at least one week at a temperature of about 4° C.
23. An aqueous preclinical formulation comprising drug substance at a concentration of about 200 mg/mL, hydroxypropyl-β-cyclodextrin at a concentration that does not exceed about 20%, wherein said drug substance is 2-(1-((1r,4r)-4-(cyanomethyl)cyclohexyl)-1,6-dihydroimidazo[4,5-d]pyrrolo[2,3-b]pyridin-2-yl)-N-(2-hydroxy-2-methylpropyl)acetamide, the formulation pH is about 2, and the aqueous preclinical formulation remains precipitate-free for at least two weeks at a temperature of about 4° C.
24. An aqueous preclinical formulation comprising drug substance at a concentration of about 200 mg/mL, hydroxypropyl-β-cyclodextrin at a concentration that does not exceed about 20%, wherein said drug substance is 2-(1-((1r,4r)-4-(cyanomethyl)cyclohexyl)-1,6-dihydroimidazo[4,5-d]pyrrolo[2,3-b]pyridin-2-yl)-N-(2-hydroxy-2-methylpropyl)acetamide, the formulation pH is about 2, and the aqueous preclinical formulation remains precipitate-free for at least three weeks at a temperature of about 4° C.
25. A method for preparing an aqueous preclinical formulation, comprising:
mixing a drug substance with a vehicle to form a drug substance-vehicle mixture,
wherein said vehicle is an aqueous solution of hydroxypropyl-β-cyclodextrin, and
wherein said drug substance is 2-(1-((1r,4r)-4-(cyanomethyl)cyclohexyl)-1,6-dihydroimidazo[4,5-d]pyrrolo[2,3-b]pyridin-2-yl)-N-(2-hydroxy-2-methylpropyl)acetamide;
   acidifying said drug substance-vehicle mixture with sufficient acid to form an acidified solution, so that the pH of said acidified solution does not exceed about 3; and
   sonicating said acidified solution.
   26. A method according to embodiment 25, wherein said hydroxypropyl-β-cyclodextrin in said aqueous solution is at a concentration that does not exceed about 20%.
   27. A method according to embodiment 25, wherein said hydroxypropyl-β-cyclodextrin in said aqueous solution is at a concentration of about 20%.
   28. A method according to embodiments 25-27, wherein said mixing of said drug substance and said vehicle is made with amounts of said drug substance and of said vehicle such that the concentration of said drug substance in said drug substance-vehicle mixture is between about 100 mg/mL and about 250 mg/mL.
   29. A method according to embodiments 25-28, wherein said mixing of said drug substance and said vehicle is made with amounts of said drug substance and of said vehicle such that the concentration of said drug substance in said drug substance-vehicle mixture is between about 100 mg/mL and about 200 mg/mL.
   30. A method according to embodiments 25-29, wherein said mixing of said drug substance and said vehicle is made with amounts of said drug substance and of said vehicle such that the concentration of said drug substance in said drug substance-vehicle mixture is about 200 mg/mL.
   31. A method according to embodiments 25-30, wherein said acidified solution pH is about 2.
   32. A method according to embodiments 25-31, wherein said acid is HCl, phosphoric acid, or sulfuric acid.
   33. A method according to embodiment 32, wherein said acid is phosphoric acid.
   34. A method according to embodiments 25-33, wherein said drug substance concentration is a target drug substance concentration.
   35. A method for preparing an aqueous preclinical formulation, comprising:
   mixing a drug substance with a vehicle to form a drug substance-vehicle mixture,
wherein said vehicle is an aqueous solution of hydroxypropyl-β-cyclodextrin, and
wherein said drug substance is 2-(1-((1r,4r)-4-(cyanomethyl)cyclohexyl)-1,6-dihydroimidazo[4,5-d]pyrrolo[2,3-b]pyridin-2-yl)-N-(2-hydroxy-2-methylpropyl)acetamide;
   acidifying said drug substance-vehicle mixture with sufficient phosphoric acid to form an acidified solution, so that the pH of said acidified solution does not exceed about 3; and sonicating said acidified solution.
   36. A method according to embodiment 35, wherein said hydroxypropyl-β-cyclodextrin in said aqueous solution is at a concentration that does not exceed about 20%.
   37. A method according to embodiment 35, wherein said hydroxypropyl-β-cyclodextrin in said aqueous solution is at a concentration of about 20%.
   38. A method according to embodiment 35, wherein said mixing of said drug substance and said vehicle is made with amounts of said drug substance and of said vehicle such that the target concentration of said drug substance in said drug substance-vehicle mixture is between about 100 mg/mL and about 250 mg/mL.
   39. A method according to embodiment 35, wherein said mixing of said drug substance and said vehicle is made with amounts of said drug substance and of said vehicle such that the target concentration of said drug substance in said drug substance-vehicle mixture is about 200 mg/mL.
   40. A method according to embodiment 35, wherein said acidified solution pH is about 2.
   41. A method according to embodiment 35, wherein said target concentration of said drug substance in said drug substance-vehicle mixture is about 200 mg/mL.
   42. A product obtained by the method of any of embodiments 25-41.
   42. A product obtainable by the method of any of embodiments 25-41.

1. An aqueous preclinical formulation comprising drug substance at a target concentration of about 100 mg/mL to about 250 mg/mL, hydroxypropyl-β-cyclodextrin at a concentration that does not exceed about 30%, wherein said drug substance is 2-(1-((1r,4r)-4-(cyanomethyl)cyclohexyl)-1,6-dihydroimidazo[4,5-d]pyrrolo[2,3-b]pyridin-2-yl)-N-(2-hydroxy-2-methylpropyl)acetamide, and the formulation pH does not exceed about 3.

2. An aqueous preclinical formulation as claimed in claim 1, wherein said hydroxypropyl-β-cyclodextrin concentration is about 15% to about 30%.

3. An aqueous preclinical formulation as claimed in claim 2, wherein said hydroxypropyl-β-cyclodextrin concentration is about 15% to about 20%.

4. An aqueous preclinical formulation as claimed in claim 1, wherein said hydroxypropyl-β-cyclodextrin concentration is does not exceed about 20%.

5. An aqueous preclinical formulation as claimed in claim 1, wherein said drug substance target concentration is about 200 mg/mL and said hydroxypropyl-β-cyclodextrin concentration does not exceed about 20%.

6. An aqueous preclinical formulation as claimed in claim 5, wherein said formulation pH is about 2.

7. An aqueous preclinical formulation as claimed in claim 1, wherein the aqueous preclinical formulation remains precipitate-free for at least one week at a temperature of about 4° C.

8. An aqueous preclinical formulation as claimed in claim 6, wherein the aqueous preclinical formulation remains precipitate-free for at least one week at a temperature of about 4° C.

9. An aqueous preclinical formulation as claimed in claim 1, wherein the aqueous preclinical formulation remains precipitate-free for at least two weeks at a temperature of about 4° C.

10. An aqueous preclinical formulation as claimed in claim 6, wherein the aqueous preclinical formulation remains precipitate-free for at least two weeks at a temperature of about 4° C.

11. An aqueous preclinical formulation as claimed in claim 1, wherein the aqueous preclinical formulation remains precipitate-free for at least three weeks at a temperature of about 4° C.

12. An aqueous preclinical formulation as claimed in claim 6, wherein the aqueous preclinical formulation remains precipitate-free for at least three weeks at a temperature of about 4° C.

13. An aqueous preclinical formulation as claimed in claim 1, wherein said hydroxypropyl-β-cyclodextrin concentration is about 20%.

14. An aqueous preclinical formulation as claimed in claim 1, wherein said hydroxypropyl-β-cyclodextrin concentration is about 20%.

15. An aqueous preclinical formulation comprising drug substance at a target concentration of about 200 mg/mL, hydroxypropyl-β-cyclodextrin at a concentration that does not exceed about 20%, wherein said drug substance is 2-(1-((1r,4r)-4-(cyanomethyl)cyclohexyl)-1,6-dihydroimidazo[4,5-d]pyrrolo[2,3-b]pyridin-2-yl)-N-(2-hydroxy-2-methylpropyl)acetamide, the formulation pH is about 2, and the aqueous preclinical formulation remains precipitate-free for at least one week at a temperature of about 4° C.

16. An aqueous preclinical formulation comprising drug substance at a target concentration of about 200 mg/mL, hydroxypropyl-β-cyclodextrin at a concentration that does not exceed about 20%, wherein said drug substance is 2-(1-((1r,4r)-4-(cyanomethyl)cyclohexyl)-1,6-dihydroimidazo[4,5-d]pyrrolo[2,3-b]pyridin-2-yl)-N-(2-hydroxy-2-methylpropyl)acetamide, the formulation pH is about 2, and the aqueous preclinical formulation remains precipitate-free for at least two weeks at a temperature of about 4° C.

17. An aqueous preclinical formulation comprising drug substance at a target concentration of about 200 mg/mL, hydroxypropyl-β-cyclodextrin at a concentration that does not exceed about 20%, wherein said drug substance is 2-(1-((1r,4r)-4-(cyanomethyl)cyclohexyl)-1,6-dihydroimidazo[4,5-d]pyrrolo[2,3-b]pyridin-2-yl)-N-(2-hydroxy-2-methylpropyl)acetamide, the formulation pH is about 2, and the aqueous preclinical formulation remains precipitate-free for at least three weeks at a temperature of about 4° C.

18. A method for preparing an aqueous preclinical formulation, comprising:
mixing a drug substance with a vehicle to form a drug substance-vehicle mixture,
wherein said vehicle is an aqueous solution of hydroxypropyl-β-cyclodextrin, and
wherein said drug substance is 2-(1-((1r,4r)-4-(cyanomethyl)cyclohexyl)-1,6-dihydroimidazo[4,5-d]pyrrolo[2,3-b]pyridin-2-yl)-N-(2-hydroxy-2-methylpropyl)acetamide;
acidifying said drug substance-vehicle mixture with sufficient phosphoric acid to form an acidified solution, so that the pH of said acidified solution does not exceed about 3; and
sonicating said acidified solution.

19. A method as claimed in claim 18, wherein said hydroxypropyl-β-cyclodextrin in said aqueous solution is at a concentration that does not exceed about 20%.

20. A method as claimed in claim 19, wherein said hydroxypropyl-β-cyclodextrin in said aqueous solution is at a concentration of about 20%.

21. A method as claimed in claim 18, wherein said mixing of said drug substance and said vehicle is made with amounts of said drug substance and of said vehicle such that the target concentration of said drug substance in said drug substance-vehicle mixture is between about 100 mg/mL and about 250 mg/mL.

22. A method as claimed in claim 21, wherein said mixing of said drug substance and said vehicle is made with amounts of said drug substance and of said vehicle such that the target concentration of said drug substance in said drug substance-vehicle mixture is about 200 mg/mL.

23. A method as claimed in claim 18, wherein said acidified solution pH is about 2.

24. A method as claimed in claim 20, wherein said acidified solution pH is about 2.

25. A method as claimed in claim 24, wherein said target concentration of said drug substance in said drug substance-vehicle mixture is about 200 mg/mL.

26. A method as claimed in claim 18, wherein
said hydroxypropyl-β-cyclodextrin in said aqueous solution is at a concentration of about 20%;
said mixing of said drug substance and said vehicle is made with amounts of said drug substance and of said vehicle such that the target concentration of said drug substance in said drug substance-vehicle mixture is about 200 mg/mL; and
said acidified solution pH is about 2.

27. A product obtained by the method claimed in claim 26.
